(19) United States
(12) Patent Application Publication
(10) Pub. No.: US 2006/0142652 A1
(43) Pub. Date: Jun. 29, 2006

(54) Concepts using the improved "composite flexible and conductive catheter electrode bands" and their method of construction

(76) Inventor: Erick Keenan, Vineland, NJ (US)

Correspondence Address: Erick Keenan, 1322 Forest Grove Road, Vineland, NJ, 08360, US

(21) Appl. No.: 11/024,107

(22) Filed: Dec. 28, 2004

(51) Int. Cl. A61B 5/04; A61N 1/05; A61N 1/06; A61B 18/18

(52) U.S. Cl. 600/374; 606/41; 607/122

(57) ABSTRACT

This invention discloses improvements in concepts in the configuration of and the utilization of a “Composite Flexible and Conductive electrode bands”. Also this document will illustrate an example method from which to construct such “Flexible” components (thermoplastic-polymer/elastomer disc) and “Conductive” components (thin metal disc) which then can be made into catheter electrode bands”. One envisioned use for the improved “Composite Flexible Conductive electrode band” is that of an electrode located at the distal tip of a cardiac catheter. The band has advantages for such an application; it has controllable flexibility due to the elastic properties of the flexible (polymer/elastomer) part and continuous uninterrupted electrical current conductance from the one-piece design of the conductive element. The synergy of the components of the improved “Composite Flexible and Conductive bands” will help solve problems current electrode bands have and will allow for a freedom in the design of catheter electrode band configurations

### CROSS REFERENCE TO RELATED APPLLICATIONS

References cited:

U.S. PATENT DOCUMENTS:

| | | |
|---|---|---|
| 5433742 | JULY, 1995 | Willis |
| 5554178 | SEPTEMBER, 1996 | Dahl et al. |
| 5558073 | SEPTEMBER, 1996 | Pomeranz et al. |
| 6032061 | FEBRUARY, 2000 | Koblish |
| 6440488 | AUGUST, 2002 | Griffin |
| 6208881 | MARCH, 2001 | Champeau |
| 6400976 | JUNE, 2002 | Champeau |
| 6456863 | SEPTEMBER, 2002 | Levin et al. |
| 6493590 | DECEMBER, 2002 | Wessman |

Other References:

Disclosure document number 532327 filed on Jun. 3, 2003.

Non-Provisional patent application number 10/367,034 filed on Feb. 19, 2003.

### REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

International Classification A61N/05, 001/05

Current United States Classification 607/122, 607/116

Field of Search 607/115, 116, 119, 122, 124, 128/642

1. Field of Invention

This invention relates to the electrophysiological catheter used in the mapping (measurement of electrical potential), pacing (stimulation of the muscle tissue by pulsing an electrical current) and ablating (burning the tissue by use of high electrical current) of the heart's inner wall. This invention generally relates to catheters and leads used in sensing electrical activity within a patient and administering therapy, and more particular to such catheters and leads incorporating band electrodes configured for flexibility and traceability within the body.

2. Discussion of Related Art

The present invention expands on the concepts presented in the Disclosure document number 532327 filed on Jun. 3, 2003 and the Non-Provisional patent application Ser. No. 10/367, 034 filed on Feb. 19, 2003. It is the duty of this document to illustrate and explain some improved embodiments of the “Composite Conductive and Flexible catheter electrode bands” as pertaining to the invention's uses, capabilities, functionality, methods of construction and other advantages they contain in use as a cardiac catheter or lead. Also, presented within the following text is evidence of various improved advantages the invention has over other prior concepts relating to their manufacturablity, performance and durability.

### BRIEF SUMMARY OF THE INVENTION

The general concept of the improved “Composite Flexible and Conductive catheter electrode band” is that each of its components uses their individual properties to perform a specific function and then they, with synergy, combine to meet the functional requirements of a 2 to 14 French diameter flexible conductive band electrode. The Flexible (polymer/elastomer) component gives the band; variable flexibility, supports the position of the conductive component, locks the conductive component safely into the band, attaches the band securely to the catheter tube, hermetically seals the conductor wire, can include a radiopaque material and can be colored to allow for visual identification of the band or give pleasing aesthetics. The conductive component (a disc shaped thin metal piece) are designed to possess an anisotrotic strength, in the desired direction and not in an undesired direction thus allowing flexibility in the required direction. The conductive component's primary functional demand is the ability to carry electrical current continuously with out interruption from the connector wire onto the surface of the electrode band contacting the patent's tissue. This invention also allows for precise band placement control down to a space of 0.010″ between conductive bands, conductive band thickness as small as 0.0005″, a method of safety locking the conductive element into the band, and a hermetically seal from fluids intrusion into the catheter inner lumens. Construction methods used to produce this invention are of a low technology in art and can be perform with simple low cost tools by technicians of moderate abilities and training. The simple design of the “Composite Flexible and Conductive electrode bands” and their attachment into a catheter and the ease of conductor wire connection lends itself to the potential to have a large degree of automated tooling to produce them. Another advantageous feature of this invention is the use of economical materials in construction of the band electrode, which are readily available from a wide source of suppliers.

While the previous statements described the band components that are the mandatory base parts to accomplish a functional “Composite Flexible and Conductive electrode band”. An added benefit of this invention is its ability to incorporated enhancements such as, a thin (0.0001″-0.0050″) encapsulation of a noble metal which when applied to the electrode's outer surface increases the electrical charge distribution and corrosion protection.

### BREIF DESCRIPTION OF THE SEVERAL VIEWS OF THE DRAWING

FIG. 1 Illustration of a catheter distal tip section view using the “Composite Flexible and Conductive bands” with “Slip tube” assembly.

FIG. 2 Illustration of a catheter distal tip section view using the “Composite Flexible and Conductive” bands.

FIG. 3 Shown is a Flexible Element.

FIG. 4 Shown is a Conductive Element.

FIG. 5 Drawing of the spacer tube.

FIG. 6 Illustration of arrangement of the elements incorporated into the “Composite Flexible and Conductive” band.

FIG. 7 Shown is a completed “Composite Flexible and Conductive” band.

FIG. 8 Shown is an exploded Illustration of the “Slip Tube” wiring device.

FIG. 9 Illustration of a catheter distal tip section view using the “Composite Flexible and Conductive bands with Noble metal encapsulation” with “Slip tube” assembly.

FIG. 10 Shown is a “Composite Flexible and Conductive bands with Noble metal encapsulation”, band is ready to use as a catheter electrode.

FIG. 11 Drawing is an enlarged break out view of the “Composite Flexible and Conductive bands with Noble metal encapsulation”.

FIG. 12 Shown is a section view of the distal tip assembly using the “Composite Flexible and Conductive bands with Noble metal encapsulation”.

FIG. 13 Shown is the “Plug” Element.

FIG. 14 Illustration is of a section view of the band with “Plugs” in place as “Co-pole”.

FIG. 15 Illustration is of a section view of the band with “Plugs” in place.

FIG. 16 Shown is a section view of the distal tip of the “Sliding Sheath” band having variable length and position catheter”.

FIG. 17 Drawing of the “Sliding Sheath ” assembly.

FIG. 18 Shown is a section view of the distal tip of the “Rolling conductive diaphragm” band, variable length catheter.

FIG. 19 Drawing of the “Rolling conductive diaphragm” band assembly.

FIG. 20 Shown is a section view of a catheter's distal tip of the “Rolling conductive diaphragm” band, variable length and position catheter.

### DETAILED DESCRIPTION OF THE INVENTION

1) Concepts using “Composite Flexible and Conductive electrode bands” 1

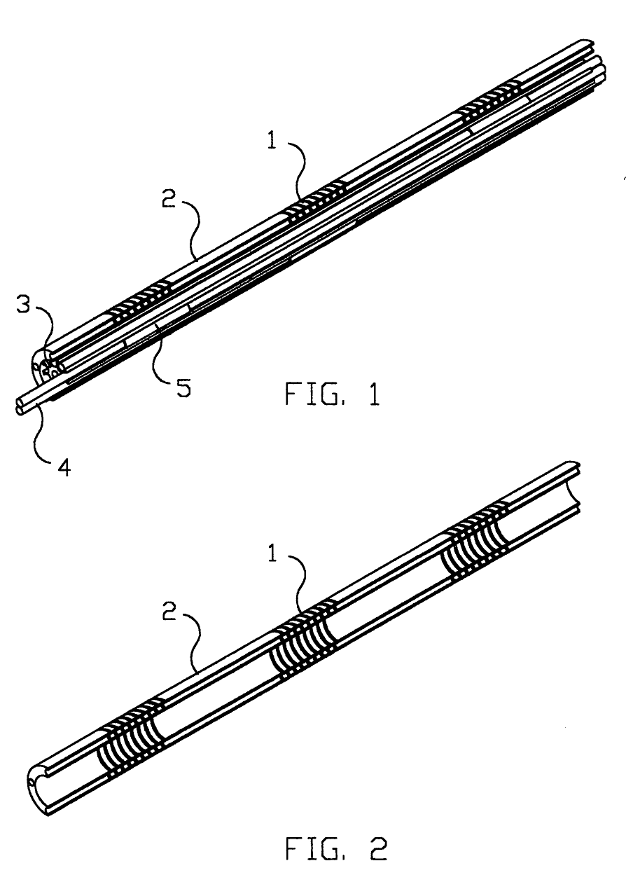

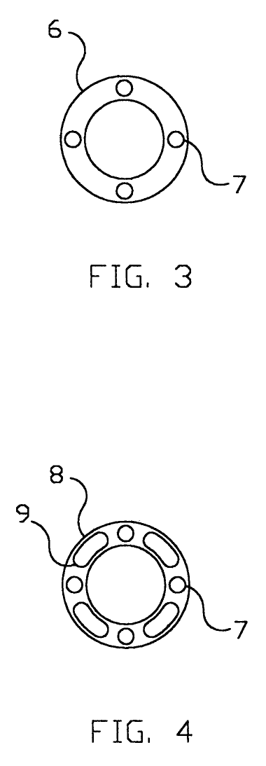

Referring to FIG. 1 is an illustration of a catheter's distal tip section view using the “Composite Flexible and Conductive” electrode bands 1. Other components which compose the assembly that are shown in FIG. 1 are the; spacer tube 2, “Slip tube” 3, conductor wire 4, and the conductive adhesive cap 5. FIG. 2 shows a section of the distal tip's outer tube assembly that is made of an arrangement of spacer tubes 2 and “Composite Flexible and Conductive” bands 1. The bands 1 are made from “Flexible components” 6 as shown in FIG. 3 and “Conductive components” 8 as shown in FIG. 4. “Flexible components” 6 can be made from any thermoplastic polymer/elastomer which may be polyurethane, PVC or any other suitable polymer. A shape as shown in FIG. 3 can be die stamped, laser cut, molded, cut from an extrude tube or by any other method used to make a disc that is 0.001″-0.250″ thick, 3-14 French outer diameter and having a inner hole diameter to what is required. The “Flexible component” 6 can be manufactured with passage holes 7, which can serve as through ways for pull wires and springs 23, see FIG. 18, for deflection mechanisms or as passage channels for fluids such as air for balloons or liquid medications. Polymer compounds use to make the “Flexible components” 6 can have a radiopaque material and/or colorant mixed into them at various concentrations depending on the requirements. The “Conductive component” 8, an example of its configuration is shown in FIG. 4, is a 0.0005″-0.1500″thick metal disc made from such metals as gold, Platinum, Silver, Stainless steel, Platinum/Iridium alloy, plated Copper, or any other suitable material. A “Conductor component” 8 can be 3-14 French in diameter disc with an Inner hole of any shape, having a pattern of safety locking holes 9 and passage holes 7 cut into it, see FIG. 4. Several methods are used to manufacture the “Conductive components” 8 and some preferred ones are listed as follows; die stamping, laser cutting or photo etching. The passage hole 7 can run from the proximal too distal, the entire length of the catheter, and have one or several tubes inserted into it. Also the passage hole 7 can contain torque control elements and also safety cables which can be an adhesive coated polymer string which holds all the components together in case of catheter failure.

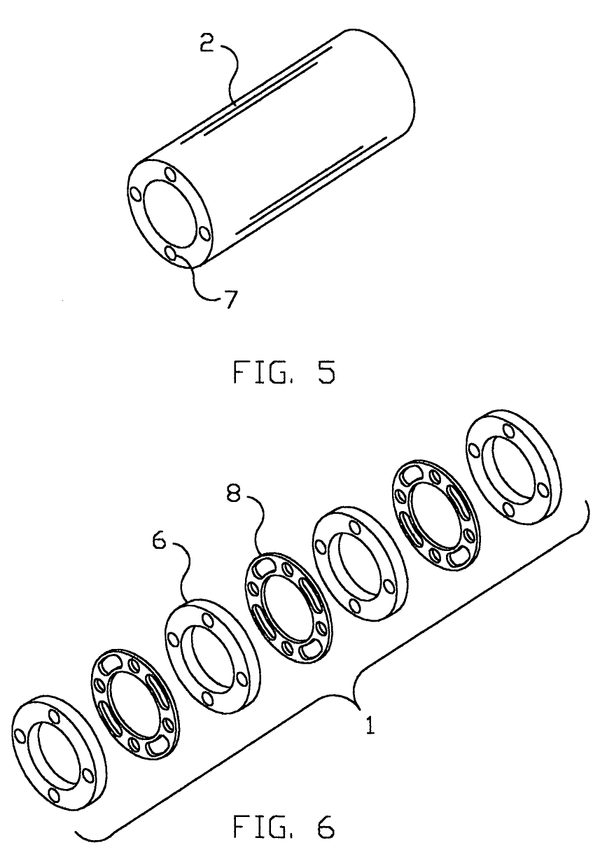

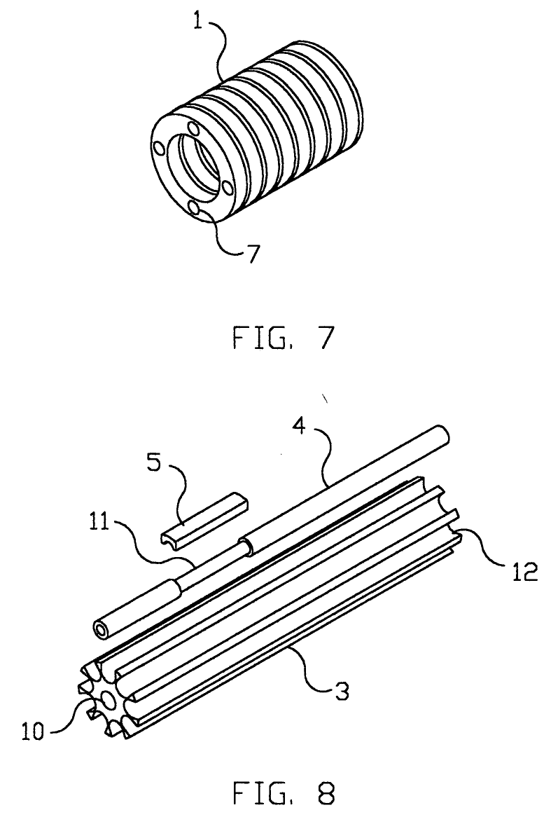

To produce an electrode band 1 an arrangement of alternating “Flexible” 6 and “Conductive” 8 discs (see FIG. 6) are stacked into a manual or automated heated press to a specified length which can be from 0.003″ to however long depending on the requirement of the distal tip. By convention the ends of the bands 1 are the “Flexible component” 6 and can be a different polymer than that which is used to make the middle ones, that is they can be formulated for more adhesion. After heating the stacked components to the softening point or glass transition temperature the pressure of the press's ram is applied to force a flowing of polymer into the safety link holes 9 area which joins the abutting flexible components 6 into a single continuous piece. Some time is allow to cool the electrode band 1 before it is removed from the press and its now ready to be joined to the distal tip for use as an electrode, see FIG. 7. At this point, “Composite Flexible and Conductive” bands 1 and spacer tubes 2 are brought together in the required arrangement and placed into a heated press to be made into a distal tip like the one shown in FIG. 2.

Electrical connection of the conductor wire 4 to the bands 1 is done by a “Slip tube” 3 device, see FIG. 8. A conductor wire 4 is stripped of its insulation down to expose the bare wire 11 and then fitted into the wire channel 12, see FIG. 8. After the exposed section of the conductor wire 11 is moved into the correct position along the “Slip tube” 3; a conductive adhesive 5 is applied over the bare wire 11 as shown in FIG. 8 and the assembly is let to cure so as to set the wire 11 firmly in place. When all of the conductor wires 4 are firmly in place the “Slip tube” 3 is now ready for insertion into the distal tip assembly to complete the catheter's electrical connections as shown in FIG. 1. Note that the center through hole 10, see FIG. 8, of the “Slip tube” 3 can be used as a air lumen, guide wire channel, or delivery port for fluids such as medications, also more than one hole with different shapes can be design into the “Slip tube” 3.

2) Concepts Using “Composite Flexible and Conductive Electrode bands with Nobel Metal Encapsulation” Band 13.

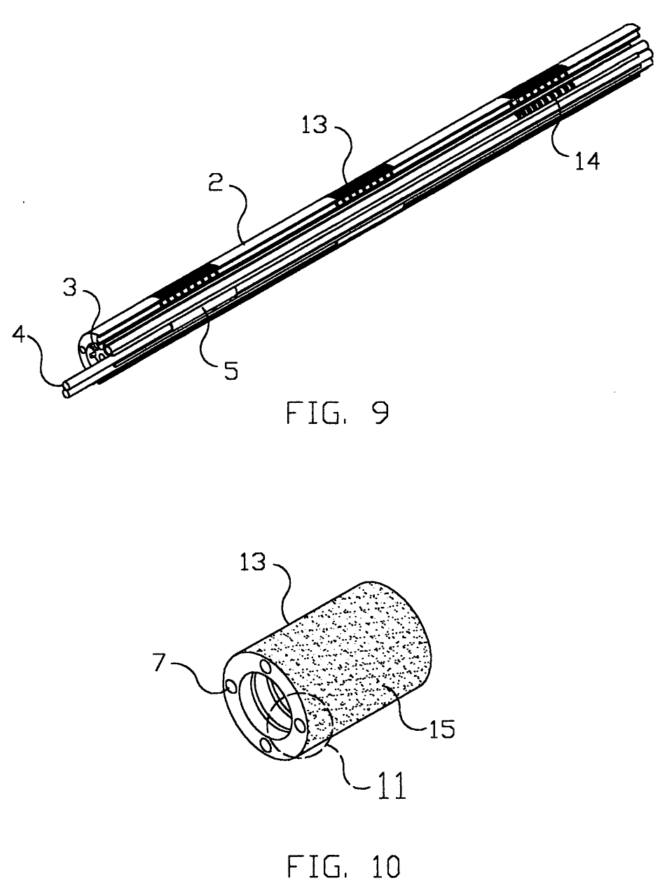

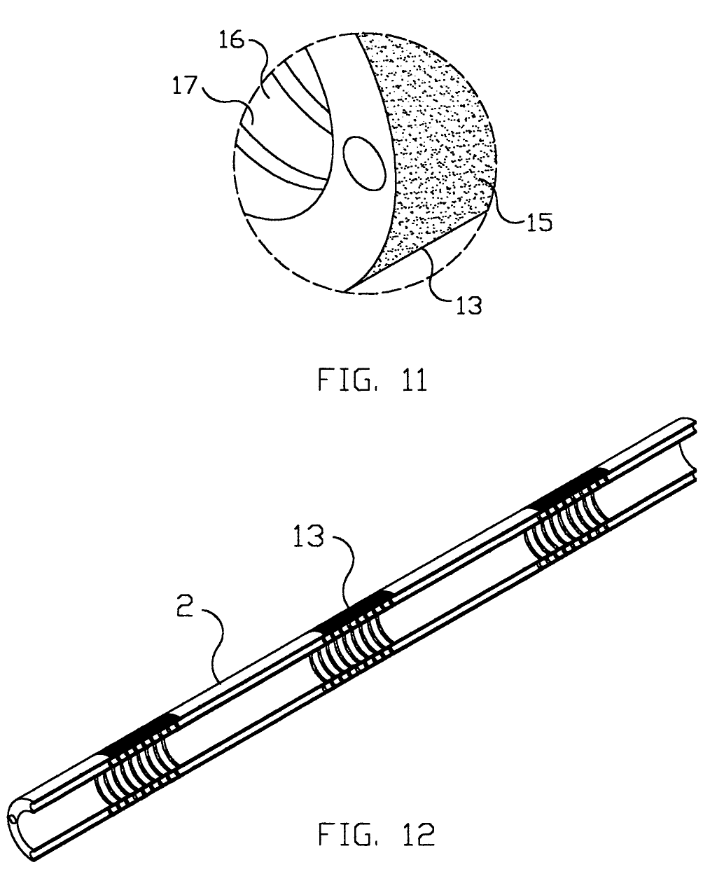

Referring to FIG. 10 is illustrated an assembled “Composite Flexible and Conductive with Nobel metal encapsulation” band 13 shown in perspective view before its assembly into a cardiac catheter tube where it performs the function of an electrode see FIG. 9. The band 13 is an assembly of; the conductive elements 8, the Flexible element 6 and a Noble metal encapsulation 15, which is shown in FIG. 11. Uninterrupted electrical currents are carried from the conductor wire end that connects to the conductor component to the outside surface of the band 13 and evenly distributed throughout the noble metal encapsulation 15. A conductive metal 16 (W, Ag, Au, Cu, Al or other) or a radiopaque material 17 can be compounded in to the Flexible component to aid in the band's visibility performance. The Nobel metal encapsulation band 15 is a 0.0001″-0.0050″ thick Noble metal coating of metal such as Pt, Au or Ag and is design to give the electrode corrosion protection and also an increased electrical charge distribution. The application of the Nobel metal can be electroplated on, vapor deposition, electroless plated and by any other efficient means applied to the outer and inner surface if so wished.

Referring to FIG. 12 is an illustration of a catheter's distal tip section view using the “Composite Flexible and Conductive with Nobel metal encapsulation” band 13. Other components which compose the assembly that are shown in FIG. 9 are the; spacer tube 2, “Slip tube” 3, conductor wire 4, and the conductive adhesive cap 5. FIG. 12 shows a section of the distal tip's outer tube assembly that is made of an arrangement of spacer tubes 2 and “Composite Flexible and Conductive with Nobel metal encapsulation” band 13.

Electrical connection of the conductor wire 4 to the bands 13 is done by a “Slip tube” 3 device, see FIG. 9. A conductor wire 4 is stripped of its insulation down to expose the bare wire 11 and then fitted into the wire channel 12, see FIG. 8. After the exposed section of the conductor wire 11 is moved into the correct position along the “Slip tube” 3; a conductive adhesive 5 is applied over the bare wire 11 as shown in FIG. 8 and the assembly is allowed to cure so as to set the wire 11 firmly in place. When all the conductor wires 4 are firmly in place the “Slip tube” 3 is now ready for insertion into the electrode band 13 and spacer tube 2 distal tip assembly (see FIG. 12) to complete the catheter's electrical connections as shown in FIG. 9. Note that the conductor over-wrap 14, see FIG. 9, is also an effective electrical contact and is a different configuration than the conductive adhesive pad 5. The over-wrap 14 is a metal wire which has been attach by twisting a metal wire tightly around the conductor's wire striped end 11.

3) Concepts for “Integrated Bands”

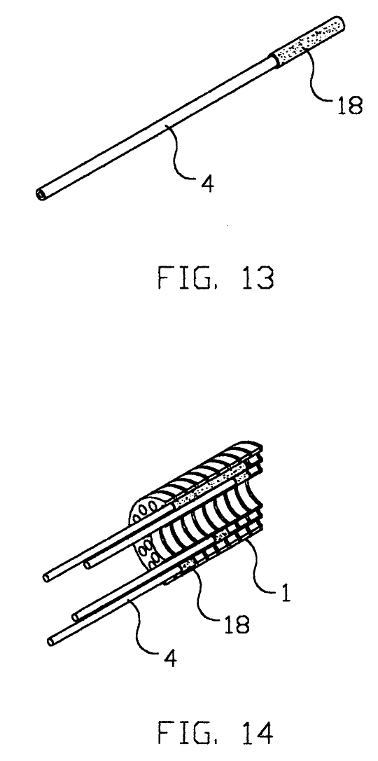

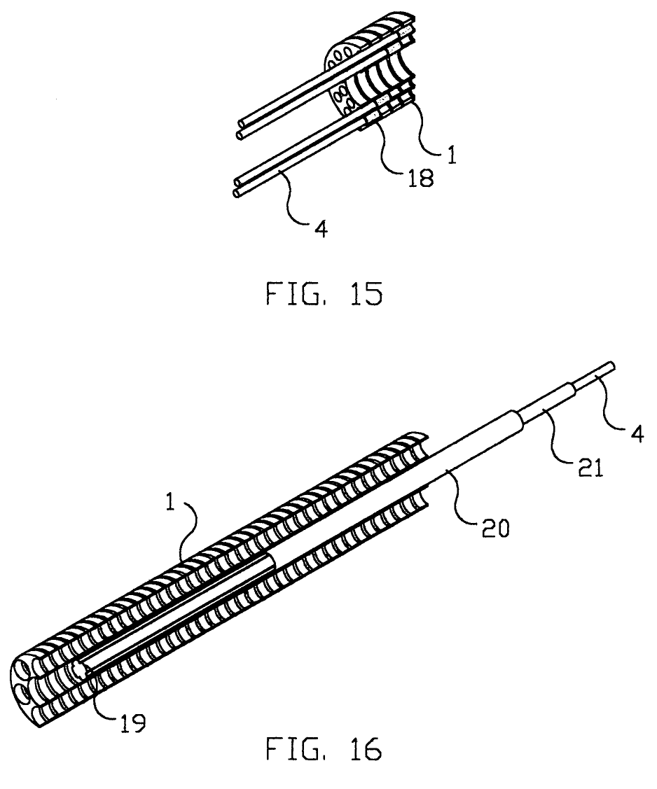

This concept envisions adapting some of the safety link holes 9 in the “Composite Flexible and Conductive electrode bands” 1 to act as through holes in which flexible conductive pads (“Plug”) 18 are placed. A “Plug” 18 is a conductor wire that has had an end striped of its insulation to a specified length and then the bare metal wire is jacked with a conductive flexible polymer tube, see FIG. 13. The Plug's 18 length may not directly correlate with band length, that is that a Plug's 18 length can be 0.010″ long and contact only one 0.002″ long conductive disc 8 creating a band length of 0.002″ (see FIG. 15) or a plug can contact as many conductive discs for a length as long as the entire distal tip (see FIG. 14). Plugs 18 can also share the same band location along the distal tip and act as “Co-pole” bands (see FIG. 14) or they can be separate as shown in FIG. 15.

4) Concepts for “Sliding Sheath” Electrode Bands of Variable Length and Position.

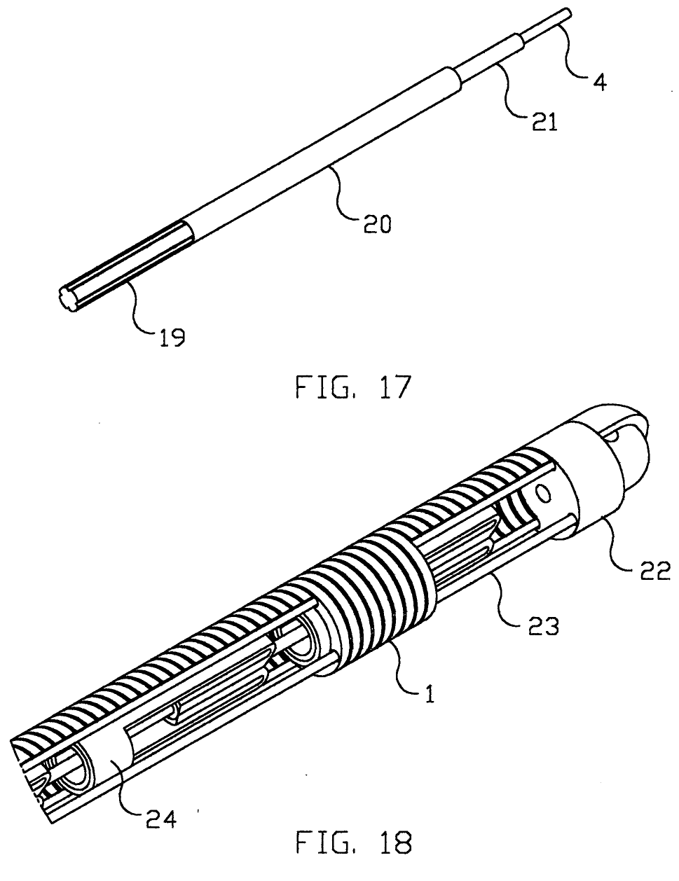

Variable length bands can be achieved from zero to the entire distal tip, see FIG.16. By activating the handle which pushes on the cover sheath tube 20 to cover the expanding conducting plug 19 or by puling the electrically insulating cover sheath tube 20 which exposes the expanding conducting plug 19 to make electrical contact to that section of “Composite Flexible and Conductive Bands” 1. Also actuation of the handle can cause a change in band position or location along the distal tip. With a push on or pull on the position actuator rod 21 (see FIG. 16) movement is provided. See FIG. 17 for an illustration of the conductive expanding plug 19, cover sheath tube 20, and position actuator rod 21 assembly.

5) Concepts for “Conductive Rolling Diaphragm” Variable Length Electrode Band.

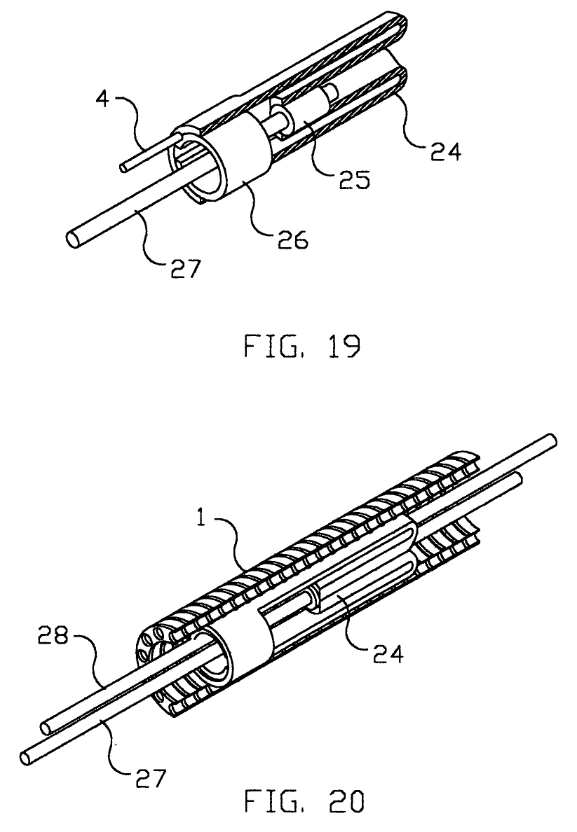

By activating the handle's knob, the pushing and pulling causes a length change in the electrode from 2 mm to what ever length is desired. Inside the distal tip is a rolling diaphragm 24 (see FIG. 18) which has a flexible conductive coating applied to the outer surface allowing it to make electrical contact between itself and the “Composite Flexible and Conductive bands” 1 (see FIG. 18). An example of its application might be of a 6 French, deflectable catheter with 7 poles 24 (See FIG. 18); note what is shown is a deflection spring cap anchor 22 and pull wires 23. FIG. 19 is a cut away section view, which illustrates some of the component parts of the conductive rolling diaphragm 24 assembly, shown is the actuator rod 27 and how it is attached to the assembly by means of a anchor tube 25. The base anchor 26 holds the position firmly in place to allow the conductive rolling diaphragm 24 to be pushed longer or pulled shorter to change contacting length.

6) Concepts for “Conductive Rolling Diaphragm” variable length and position electrode band.

By activating the handle, the bands length are changed as described in the text above but, now an added feature has been incorporated into the catheter which allows for the band's location along the distal tip to change (see FIG. 20). Added is the position actuator rod 28, which pushes or pulls the connected Conductive rolling diaphragm (s) 24 to change their position along the distal tip.

It should be noted that, as shown in FIG. 18, the “Conductive Rolling Diaphragm” system could be a single unit or multiple.

1. I claim the invention of the improved “Composite Flexible and Conductive electrode band” comprised of the following.
a. The “Flexible component” is made of a 0.001″-0.250″ thick thermoplastic polymer/elastomer such as, for example; polyurethane, PVC, nylon which is made into a round 2-14 French diameter disc with a through hole or holes.
b. The “Flexible component” can have a radiopaque material and/or colorant mixed into the polymer at various concentrations depending on the application.
c. The “Conductive component” is a 0.0005″-0.1500″ thick metal, such as: Gold, Platinum, Silver, Stainless Steel, Platinum/Iridium alloy, plated copper, disc shaped, 2-14 French outer diameter disc with a pattern of safety locking link holes and other utility holes spaced through it.

2. I claim the invention of an improved method of construction of the improved “Composite Flexible and Conductive electrode band”, which is performed as follows.
a. Die stamp, mold, extrude or by any other effective method, manufacture the “Flexible component” to the prescribed design specifications in the required numbers.
b. Die stamp, laser cut, photo etched or by any other effective method, manufacture the “Conductive component” to the prescribed design specifications in the required numbers.
c. An arrangement of alternating “Flexible” and “Conductive” components are stacked into a heated press to a length specified which can be from 0.003″ to how ever long depending on the requirements.
d. The ends of the stack are conventionally the “Flexible component” and can be made of a different polymer than the other “Flexible components”.
e. After heating the “Flexible components” to the polymer's softening point and applying the pressure of the press's ram, flowing polymer will fill into the link hole area and join with the abutting “Flexible components” to form a continuous piece; after cooling and removal from the press the band is ready for use.

3. I claim the invention of the “Slip tube” wiring system with center through hole to make electrical contact between the improved “Composite Flexible and Conductive electrode band” used as a catheter's distal tip electrodes and the conductor wires.
a. The extruded or molded polymer “Slip tube” holds the conductor wires in position along the catheter tube body and in contact with the improved “Composite Flexible and Conductive electrode band”.
b. The “Slip tube” can be made from a single polymer (material) or a composite to give different properties along its length.

4. I claim the invention of the improved “Composite Flexible and Conductive electrode band” in its use as a cardiac catheter electrode as described in the following method of construction for that use.
a. A predetermined arrangement of a detached stack of spacer tubes and “Composite Flexible and Conductive electrode band” components are placed into a heated press tool that will transform the group into catheter's distal tip and an array of electrodes.
b. The completion of the catheter is done when the “Slip tube” is positioned into a placement causing electrical contact between the electrodes and the conductor wires.

5. I claim the invention of the use of a noble metal encapsulation, such as Platinum, of (0.0001″-0.0050″) in thickness onto the outer surface of the “Composite Flexible and Conductive electrode band” to enhance its electrical properties, give corrosion resistance and other benefits that may occur.
a. The noble metal can be applied by use of electrically plating, vapor deposition or an electrodeless solution.

6. I claim the invention of “Integrated Bands” using the improved “Composite Flexible and Conductive electrode band” with a multiple hole configuration to allow for the connection of multiple “plug” conductor wires to electrically attach into an electrode, thus sharing that band.
a. The use of “plugs” is also another way that the connection of conductor wires and electrode bands can occur.

7. I claim the invention of a “Sliding Sheath” electrode band capable of variable length and position that can travel along a catheter's distal tip comprised of the improved “Composite Flexible and Conductive electrode band”.
a. Variable length bands can be achieved from zero to the entire distal tip.
b. By activating the handle which pushes on the cover sheath tube to cover the expanding conducting plug or by puling the cover sheath tube which exposes the expanding conducting plug to make electrical contact to that section of “Composite Flexible and Conductive Bands”.
c. Actuation of a handle can cause a change in band position or location along the distal tip, with a push on or pull on the position actuator rod movement is provided
d. An electrical insulating “Sliding Sheath” that covers the expanding conducting plug is actuated to decrease or increase electrode band length.

8. I claim the invention of a “Rolling Conductive diaphragm” electrode band capable of variable length and position that can travel along a distal tip comprised of the improved “Composite Flexible and Conductive electrode band”.
a. The diaphragm can be made from conductive flexible material such as a electrically conducting polymer or thin sheet metal that can be manipulated to “roll up” or “roll out” to change the length of the electrode band.

9. I claim the invention of a conjoined group “Rolling Conductive diaphragm” electrode bands with the capabilities of varying length and position along a catheter's distal tip comprised of the improved “Composite Flexible and Conductive electrode band” which can number 2 to 35 electrode bands.
